(19) United States
(12) Patent Application Publication
(10) Pub. No.: US 2024/0002766 A1
(43) Pub. Date: Jan. 4, 2024

(54) MICROENVIRONMENT-SIMULATED CELL CULTURE SYSTEM

(71) Applicant: NATIONAL TSING HUA UNIVERSITY, HSINCHU (TW)

(72) Inventors: Hsuan-Yu MU, Hsinchu (TW); Jen-Huang HUANG, HSINCHU (TW)

(21) Appl. No.: 18/061,322

(22) Filed: Dec. 2, 2022

(30) Foreign Application Priority Data
Jun. 30, 2022 (TW) 111124575

(51) Int. Cl. C12M 1/12; C12M 1/00

(52) U.S. Cl. CPC C12M 25/06; C12M 23/22; C12M 29/12

(57) ABSTRACT

A microenvironment-simulated cell culture system includes a cell culture chip, a fluid storage device and a fluid driving member. The cell culture chip includes a mainbody, a cell culture chamber, two fluid delivery ports and a sample loading well. The cell culture chamber is disposed in the mainbody and includes a first side portion and a second side portion. The two fluid delivery ports are separately disposed on the mainbody and respectively connected to the cell culture chamber. The sample loading well is disposed on the mainbody and connected to the cell culture chamber. The fluid storage device is pipe-connected to the cell culture chip. The fluid driving member is pipe-connected to the fluid storage device and the cell culture chip.

### RELATED APPLICATIONS

This application claims priority to Taiwan Application Serial Number 111124575, filed Jun. 30, 2022, which is herein incorporated by reference.

### BACKGROUND

### Technical Field

The present disclosure relates to a cell culture system. More particularly, the present disclosure relates to a microenvironment-simulated cell culture system which can simulate the microenvironment during cell growth.

### Description of Related Art

In the modern society, cancer has brought a great threat to the safety of human life, and thus how to effectively detect cancer early and take the appropriate treatment are important research goals in the current clinical practice.

The screening of anticancer drugs is mostly achieved by two-dimensional cell culture, three-dimensional cell spheroid culture or animal experiments. However, in the two-dimensional cell culture, the growth of the cancer cells can only be observed on surfaces, and it cannot represent the actual complexity of the tumor microenvironment. Further, the characteristics of the cell diversity and the richness of the extracellular matrix in tumors also cannot be simulated, resulting in the drug screening results are not consistent with the actual pharmacological effects. Furthermore, in the three-dimensional cell spheroid culture, although the tissue characteristics in tumors can be simulated, but the gradient of oxygen, the nutrient distribution and the distribution of immune cells are not easy to observe in the cell spheroid. Moreover, by performing animal experiments, the time and the cost for screening anticancer drugs may be extremely increased, and the reproducibility of the experiment is not as good as expected.

Therefore, how to improve the in vitro cell culture device so as to accurately simulate the microenvironment of cell growth in tumors and then screen anticancer drugs for different types of cancer or develop new treatment guidelines has become the aim of modern practitioners and academics.

### SUMMARY

According to one aspect of the present disclosure, a microenvironment-simulated cell culture system includes a cell culture chip, a fluid storage device and a fluid driving member. The cell culture chip includes a mainbody, a cell culture chamber, two fluid delivery ports and a sample loading well. The cell culture chamber is disposed in the mainbody and includes a first side portion and a second side portion, wherein the first side portion and the second side portion are respectively disposed on two ends of the cell culture chamber along a long axis of the mainbody. The two fluid delivery ports are separately disposed on the mainbody and respectively connected to the cell culture chamber. The sample loading well is disposed on the mainbody and connected to the cell culture chamber. The fluid storage device is pipe-connected to the cell culture chip, wherein the fluid storage device is connected to the cell culture chamber by one of the fluid delivery ports. The fluid driving member is pipe-connected to the fluid storage device and the cell culture chip, wherein the fluid driving member is connected to the cell culture chamber by the other one of the fluid delivery ports. The cell culture chamber is substantially a long-stripped slot, two long sides of the cell culture chamber are parallel to the long axis of the mainbody, and a length ratio of a short side of the cell culture chamber to one of the long sides of the cell culture chamber is 1:1 to 1:4.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiment, with reference made to the accompanying drawings as follows:

FIG. 1 is a schematic view of a microenvironment-simulated cell culture system according to one embodiment of the present disclosure.

FIG. 2 is a schematic view of a cell culture chip of the microenvironment-simulated cell culture system of FIG. 1.

FIG. 3 is an exploded view of the cell culture chip of FIG. 2.

FIG. 4 shows the results of Western blotting analysis of the co-cultivation of 4T1 cells and K-BALB cells for 24 hours by the microenvironment-simulated cell culture systems of Example 1 to Example 3.

FIG. 5 shows the analysis results of hypoxia signal quantification in different areas of the cell culture chamber of the microenvironment simulated cell culture system of Example 2.

FIG. 6 shows the analysis results of cell viability in different areas of the cell culture chamber after culturing for 24 hours with different drug combinations.

FIG. 7A shows the staining results of the propidium iodide in different areas of the cell culture chamber of Testing example 1.

FIG. 7B shows the staining results of the propidium iodide in Area 4 of the cell culture chambers of Testing example 1 to Testing example 4.

FIG. 8 shows the quantification results of mRNA expression in the normoxia area and the hypoxia area of the cell culture chamber of Testing example 1.

FIG. 9A shows the quantification results of mRNA expression in the normoxia areas of the cell culture chambers of Testing example 1 and Testing example 3.

FIG. 9B shows the quantification results of mRNA expression in the hypoxia areas of the cell culture chambers of Testing example 1 and Testing example 3.

FIG. 10A shows the quantification results of mRNA expression of the K-BALB cells in the normoxia areas of the cell culture chambers of Control group 2 and Testing example 4 to Testing example 6.

FIG. 108 shows the quantification results of mRNA expression of the K-BALB cells in the hypoxia areas of the cell culture chambers of Control group 2 and Testing example 4 to Testing example 6.

FIG. 11A shows the quantification results of mRNA expression of 4T1 cells in the normoxia areas of the cell culture chambers of Control group 3, Testing example 7 and Testing example 8.

FIG. 11B shows the quantification results of rnRNA expression of 4T1 cells in the hypoxia areas of the cell culture chambers of Control group 3, Testing example 7 and Testing example 8.

FIG. 12A shows the analysis results of the percentage of T cells expressing Tim-3 receptor in the normoxia area and the hypoxia area of the cell culture chamber.

FIG. 12B shows the analysis results of the percentage of T cells expressing CTLA-4 receptor in the normoxia area and the hypoxia area of the cell culture chamber.

FIG. 12C shows the analysis results of the percentage of T cells expressing PD-1 receptor in the normoxia area and the hypoxia area of the cell culture chamber.

FIG. 13 shows the staining results of T cells in different areas of the cell culture chamber after being cultured for 4 hours and 24 hours.

FIG. 14 shows the staining results of apoptosis signaling-related proteins in different areas of the cell culture chamber.

FIG. 15A shows the analysis results of the ratio of T cells to the total cell mass in the hypoxia area of the cell culture chamber after being treated with different doses of anti-PD-1 antibody.

FIG. 15B shows the analysis results of the ratio of T cells to the total cell mass in the normoxia area of the cell culture chamber after being treated with different doses of anti-PD-1 antibody.

FIG. 16A shows the analysis results of the percentage of apoptosis in the hypoxia area of the cell culture chamber after being treated with different doses of anti-PD-1 antibody.

FIG. 16B shows the analysis results of the percentage of apoptosis in the normoxia area of the cell culture chamber after being treated with different doses of anti-PD-1 antibody.

FIG. 17A shows the analysis results of the ratio of T cells to the total cell mass in the hypoxia area of the cell culture chamber after being treated with different drugs.

FIG. 17B shows the analysis results of the ratio of T cells to the total cell mass in the normoxia area of the cell culture chamber after being treated with different drugs.

FIG. 18A shows the analysis results of the percentage of apoptosis in the hypoxia area of the cell culture chamber after being treated with different drugs.

FIG. 18B shows the analysis results of the percentage of apoptosis in the normoxia area of the cell culture chamber after being treated with different drugs.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

[The Microenvironment-Simulated Cell Culture System of the Present Disclosure]

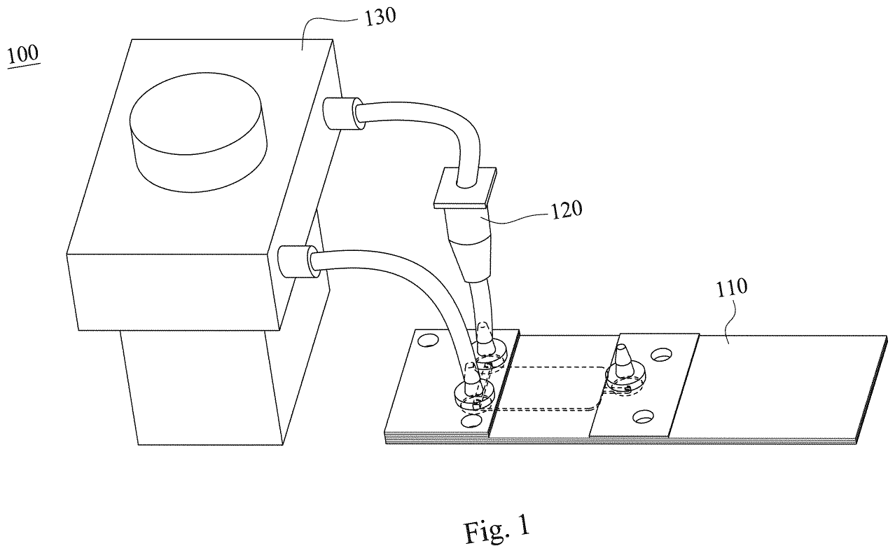

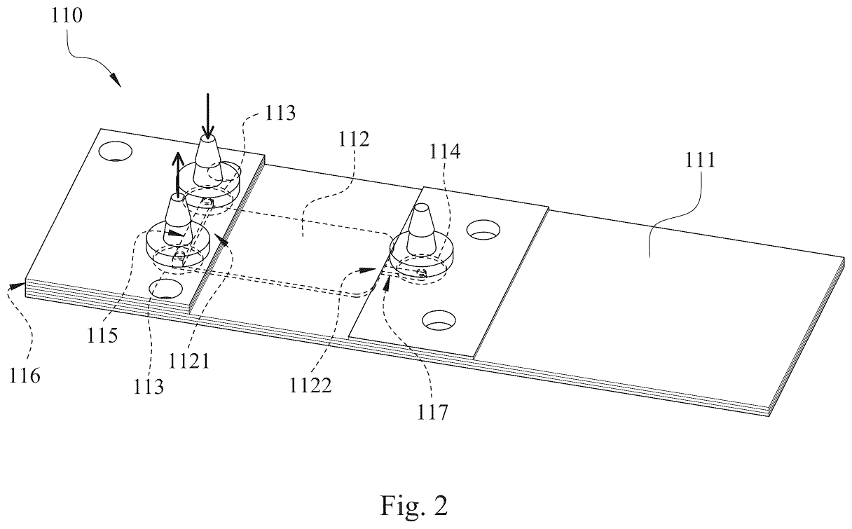

Reference is made to FIG. 1 and FIG. 2, wherein FIG. 1 is a schematic view of a microenvironment-simulated cell culture system 100 according to one embodiment of the present disclosure, and FIG. 2 is a schematic view of a cell culture chip 110 of the microenvironment-simulated cell culture system 100 of FIG. 1. The microenvironment-simulated cell culture system 100 includes a cell culture chip 110, a fluid storage device 120 and a fluid driving member 130.

The cell culture chip 110 includes a mainbody 111, a cell culture chamber 112, two fluid delivery ports 113 and a sample loading well 114.

The cell culture chamber 112 is disposed in the mainbody 111. The cell culture chamber 112 includes a first side portion 1121 and a second side portion 1122, the first side portion 1121 and the second side portion 1122 are respectively disposed on two ends of the cell culture chamber 112 along a long axis (not shown) of the mainbody 111. In particular, in the embodiment of FIG. 1, the mainbody 111 is substantially a rectangle, the cell culture chamber 112 is substantially a long-stripped slot, and the first side portion 1121 and the second side portion 1122 are respectively located in the cell culture chamber 112 and are near two end portions of the mainbody 111. Furthermore, as shown in FIG. 2, two long sides of the cell culture chamber 112 are parallel to the long axis of the mainbody 111, and a length ratio of a short side of the cell culture chamber 112 to one of the long sides of the cell culture chamber 112 is 1:1 to 1:4. Thus, it is favorable for establishing a molecular gradient in the cell culture chamber 112 when the cells are cultured therein. Preferably, the length ratio of the short side of the cell culture chamber 112 to the one of the long sides of the cell culture chamber 112 can be 1:2. Moreover, a height of the cell culture chip 110 can be 0.25 mm to 0.75 mm, and the present disclosure is not limited thereto.

The two fluid delivery ports 113 are separately disposed on the mainbody 111, and the two fluid delivery ports 113 are respectively connected to the cell culture chamber 112 so as to transport the fluid in the cell culture chamber 112. Further, the two fluid delivery ports 113 can be separately disposed along a direction parallel to the short side of the cell culture chamber 112 so as to establish the fluid circulation subsequently, and the present disclosure is not limited thereto.

The sample loading well 114 is disposed on the mainbody 111, and the sample loading well 114 is connected to the cell culture chamber 112 so as to transport the cells to be cultured into the cell culture chamber 112.

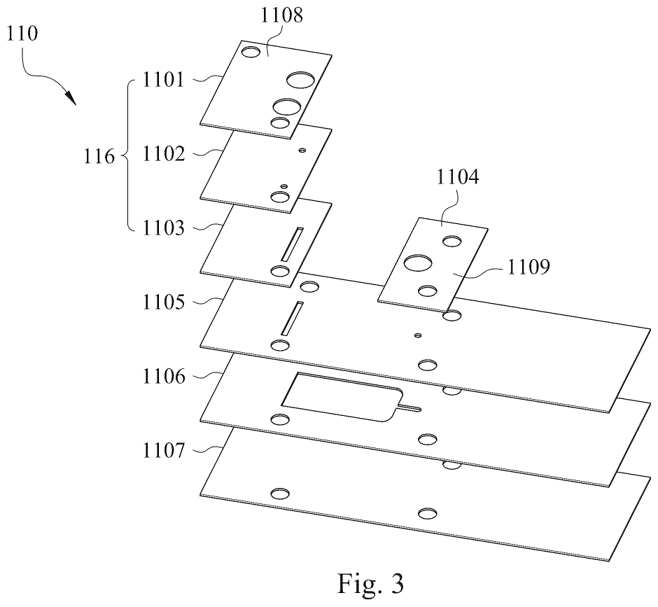

Reference is made to FIG. 2 and FIG. 3 simultaneously, wherein FIG. 3 is an exploded view of the cell culture chip 110 of FIG. 2. As shown in FIG. 3, the cell culture chip 110 is a multi-layers structure and includes a first base plate 1101, a second base plate 1102, a third base plate 1103, a fourth base plate 1104, a fifth base plate 1105, a sixth base plate 1106 and a seventh base plate 1107.

As shown in FIG. 2 and FIG. 3, the first base plate 1101 has a first surface 1108, and the two fluid delivery ports 113 are separately opened on the first surface 1108 (reference number is shown in FIG. 3), wherein the first base plate 1101, the second base plate 1102, the third base plate 1103 and the fifth base plate 1105 are stacked in sequence to form a fluid channel 115 (reference number is shown in FIG. 2), and the fluid channel 115 is connected to the two fluid delivery ports 113 and the cell culture chamber 112. Further, the first base plate 1101, the second base plate 1102 and the third base plate 1103 are stacked in sequence to form a first covering unit 116, and the first covering unit 116 covers the first side portion 1121. Therefore, by the arrangements that the two fluid delivery ports 113 are opened on the first surface 1108 of the first base plate 1101, and the first covering unit 116 formed by the first base plate 1101, the second base plate 1102 and the third base plate 1103 covers the first side portion 1121, the fluid input from one of the fluid delivery ports 113 can be transported into the cell culture chamber 112 by the fluid channel 115 and then transported out of the cell culture chamber 112 by the other one of the fluid delivery ports 113. Therefore, it is favorable for effectively simulating the material exchange between the tumor and the external environment thereof, and the interaction between the tumor microenvironment and blood vessels, such as the shear force and the normal force caused by the flow of the blood and the tissue fluid to the tumor tissue, etc., also can be simulated. Thus, it has excellent clinical application potential.

As shown in FIG. 2 and FIG. 3, the fourth base plate 1104 has a second surface 1109 (reference number is shown in FIG. 3), and the sample loading well 114 is opened on the second surface 1109. The fourth base plate 1104 and the fifth base plate 1105 are stacked in sequence to form a loading channel 117 (reference number is shown in FIG. 2), the loading channel 117 is connected to the sample loading well 114 and the cell culture chamber 112, and the fourth base plate 1104 can cover the second side portion 1122, or be disposed adjacent to the second side portion 1122 to cover the loading channel 117. Therefore, by the arrangements that the sample loading well 114 is opened on the second surface 1109 of the fourth base plate 1104, and the fourth base plate 1104 covers the second side portion 1122 or is disposed adjacent to the second side portion 1122, the suspension including the cells to be cultured can be transported into the cell culture chamber 112 by the sample loading well 114 so as to facilitate the cells to adhere and then grow in the three-dimensional space of the cell culture chamber 112. Further, if the extracellular matrix fluid (such as collagen) is fully mixed with the cell suspension and then transported into the cell culture chamber 112 through the sample loading well 114, not only the cells can grow in the three-dimensional space of the cell culture chamber 112, but also the states that the tumor is rich in the extracellular matrix and the hyperplasia of the connective tissues can be simulated, and the present disclosure is not limited thereto.

Furthermore, as shown in FIG. 2 and FIG. 3, the fifth base plate 1105, the sixth base plate 1106 and the seventh base plate 1107 are stacked in sequence to form the cell culture chamber 112. Therefore, the assembling margin of the cell culture chip 110 can be effectively enhanced, so that the overall structure thereof can be more stable.

Further, the first base plate 1101, the second base plate 1102, the third base plate 1103, the fourth base plate 1104, the fifth base plate 1105, the sixth base plate 1106 and the seventh base plate 1107 can be made of an impermeable material. In particular, by the arrangement that the cell culture chip 110 is formed by stacking a plurality of impermeable base plates, the areas where the cell culture chamber 112 can communicate with a chip-external space are restricted to the two fluid delivery ports 113 on the first side portion 1121 and the sample loading well 114 on the second side portion 1122. Furthermore, after the cells to be cultured are transported to the cell culture chamber 112 by the sample loading well 114, the sample loading well 114 will be closed. At this time, the areas where the substances can be exchanged between the cell culture chamber 112 and the external space are only the two fluid delivery ports 113, so that it is favorable for establishing the molecular gradient along the first side portion 1121 to the second side portion 1122 of the cell culture chamber 112, and the molecular gradient can influence the cell growth during the cultivation of cells subsequently. Furthermore, the impermeable material can be polyethylene terephthalate, acrylic, polycarbonate, polystyrene or glass, but the present disclosure is not limited thereto. Moreover, the impermeable material can be a transparent material so as to facilitate the direct observation thereof and then enhance the convenience of use.

The fluid storage device 120 is pipe-connected to the cell culture chip 110, and the fluid storage device 120 is connected to the cell culture chamber 112 by one of the fluid delivery ports 113.

The fluid driving member 130 is pipe-connected to the fluid storage device 120 and the cell culture chip 110, and the fluid driving member 130 is connected to the cell culture chamber 112 by the other one of the fluid delivery ports 113.

In particular, the fluid storage device 120 and the fluid driving member 130 are respectively connected to the cell culture chamber 112 by different fluid delivery ports 113, and the fluid storage device 120 is for storing a cell culture medium. The fluid driving member 130 is for continuously driving the cell culture medium to be transferred from the fluid storage device 120 to the cell culture chamber 112 through the one of the fluid delivery ports 113 and then be removed from the cell culture chamber 112 through the other one of the fluid delivery ports 113, and the cell culture medium will continue to circulate and flow along this path so as to establish a dynamic fluid circulation system in the cell culture chip 110. Thus, the interaction between tumors and the circulatory system in the organism can be simulated so as to apply in the subsequent applications.

Furthermore, the fluid driving member 130 can be a peristaltic pump. The peristaltic pump can transport the liquid by pressing and releasing the peristaltic tubes (not shown) thereof by turns, and the liquid therein can be isolated within the peristaltic tubes without contact with other elements of the peristaltic pump. Accordingly, the peristaltic pump has advantages of low contaminate rate and continuous fluid delivery. Therefore, it is favorable for the microenvironment-simulated cell culture system 100 of the present application to screen the anticancer drugs without being affected by external substances, and thus the microenvironment-simulated cell culture system 100 of the present application has the potential for clinical application.

Therefore, by the method that the cells are cultured in the three-dimensional space of the cell culture chamber 112 of the cell culture chip 110, a three-dimensional growing tumor model can be created in the microenvironment-simulated cell culture system 100 of the present disclosure in a short period of time. Further, by the arrangement that the cell culture chamber 112 is a long-stripped slot and the first side portion 1121 and the second side portion 1122 are respectively disposed on the two ends of the cell culture chamber 112, the area where the substances can be exchanged between the cell culture chamber 112 and the external space is restricted to the first side portion 1121. At the same time, the fluid circulation system established by the fluid driving member 130 communicates the first side portion 1121 and flows cyclically, so that the oxygen, nutrients and other substances in the cell culture chamber 112 can be exchanged at the first side portion 1121, and a molecular gradient that gradually decreases along a direction from the molecular gradient to the second side portion 1122 can be established in the cell culture chamber 112. Thus, it is favorable for more accurately simulating the conditions of oxygen, nutrients and immune cells in tumor clinically, so that the anticancer drugs for different types of cancer can be screened, and the in vivo process of immune cells fighting to the tumors can be simulated. Accordingly, the time required for the conventional experiments can be greatly shortened, and the tests thereof have high reproducibility and potential for clinical application.

### EXAMPLES

The simulating effects of the actual tumor microenvironment of the microenvironment-simulated cell culture system of the present disclosure will be further exemplified by performing the cell cultivation with the microenvironment-simulated cell culture system of the present disclosure, and the experiments will be further conducted with different drugs or immune cells. However, the readers should understand that the present disclosure should not be limited to these practical details thereof, that is, in some embodiments, these practical details are used to describe how to implement the materials and methods of the present disclosure and are not necessary.

The following experiments are performed by the microenvironment-simulated cell culture system of the present disclosure. In the experiments, the cell culture chamber of the cell culture chip is equally divided into a normoxia area and an hypoxia area along a direction from the first side portion to the second side portion, wherein the normoxia area is further equally divided into Area 1 and Area 2 along the direction from the first side portion to the second side portion, the hypoxia area is also further equally divided into Area 3 and Area 4 along the direction from the first side portion to the second side portion, and the order of the oxygen concentration is: Area 1>Area 2>Area 3>Area 4. Further, a transition zone between the normoxia area and the hypoxia area is defined by that Area 2 and Area 3 are respectively divided into two regions, and the two adjacent regions of Area 2 and Area 3 are the transition zone.

The following tests are respectively performed by co-culturing 4T1 mouse breast cancer cells (“4T1 cells” hereafter) and the K-BALB fibroblasts (“K-BALB cells” hereafter) in the microenvironment-simulated cell culture systems of Example 1 to Example 3, wherein 4T1 cells belong to a triple-negative breast cancer cell line and are often used as a research model for distant metastasis of breast cancer and a clinical drug screening model, and K-BALB cells is a fibroblast cell line homologous to 4T1 cells. Further, the cell culture medium for culturing 4T1 cells is the 89% high glucose DMEM including 10% fetal bovine serum (FBS) and 1% Penicillin/Streptomycin solution (P/S), and the cell culture medium for culturing K-BALE cells is the 89% high glucose DMEM including 10% bovine calf serum and 13% Penicillin/Streptomycin solution. 4T1 cells and K-BALB cells are co-cultured at 37° C., 5% CO₂ for 24 hours so as to carry out different analysis, and the growth statuses of 4T1 cells and K-BALE cells are observed and the molecular expression in the areas with different oxygen concentrations are analyzed.

Further, in the following tests, a length ratio of a short side to a long side of the cell culture chamber of the microenvironment-simulated cell culture system of Example 1 is 1:4, a length ratio of a short side to a long side of the cell culture chamber of the microenvironment-simulated cell culture system of Example 2 is 1:2, and a length ratio of a short side to a long side of the cell culture chamber of the microenvironment-simulated cell culture system of Example 3 is 1:1. Furthermore, the cell culture chip, the fluid storage device and the fluid driving member of each of the microenvironment-simulated cell culture systems of Example 1 to Example 3 are the same as that of the microenvironment-simulated cell culture system 100 of FIG. 1, so that the details of the same structures or the arrangements are shown in the aforementioned paragraphs and will not be described herein again.

I. 4T1 Cells and K-BALB Cells are Cultured in the Microenvironment-Simulated Cell Culture System of the Present Disclosure

In the present test, the cell suspensions including 4T1 cells and K-BALB cells are respectively transported into the cell culture chamber through the sample loading well of each of the microenvironment-simulated cell culture systems of Example 1 to Example 3 first, and then the sample loading well is closed so as to facilitate the attachment and growth of 4T1 cells and K-BALB cells in the cell culture chamber. At the same time, the fluid driving member will drive the cell culture medium in the fluid storage device transporting through one of the fluid delivery ports to the cell culture chamber and then moving out of the cell culture chamber through the other one of the fluid delivery ports, and the cell culture medium will continue to flow and circulate along the aforementioned path so as to establish a dynamic fluid circulation system in the cell culture chip. Next, the microenvironment-simulated cell culture systems of Example 1 to Example 3 will be maintained under 37° C., 5% CO₂ for 24 hours so as to facilitate the three-dimensional growth of 4T1 cells and K-BALB cells and then process the subsequent analysis.

Furthermore, it is noted that if the methods and details of the following experiments are known in the art, they will not be described in detail.

II. Analysis of the Oxygen Concentration Gradient in the Cell Culture Chip

In the present test, the expressions of the hypoxia-inducible factor 1-alpha (“HIF 1-α” hereafter) in different areas of the microenvironment-simulated cell culture systems of Example 1 to Example 3 are analyzed. In detail, HIF 1-α is a transcription factor in the cellular environment that is activated in the conditions of oxygen reduction or hypoxia. If the expression of HIF 1-α is higher, the oxygen concentration in the area is lower. Thus, the protein expressions of HIF 1-α of the cells in different areas are further analyzed by Western blotting method, and the cells cultured in the microenvironment-simulated cell culture systems of Example 1 to Example 3 are stained with the fluorescent dye of Invitrogen™ Image-iT™ Red Hypoxia Reagent so as to assess whether the oxygen concentration gradient is established in the cell culture chamber or not.

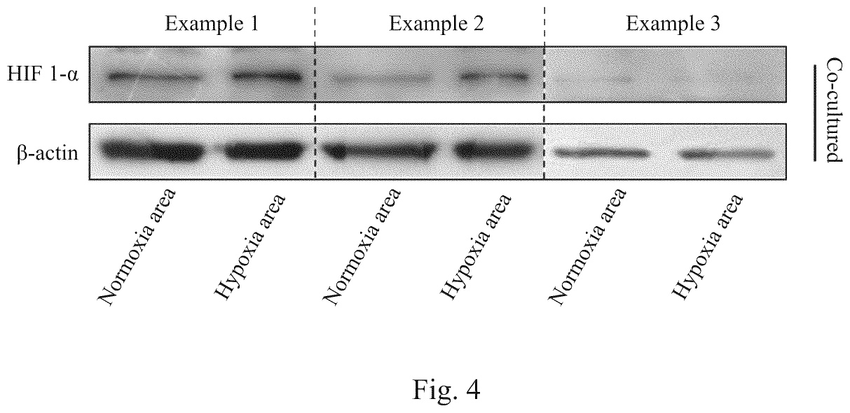

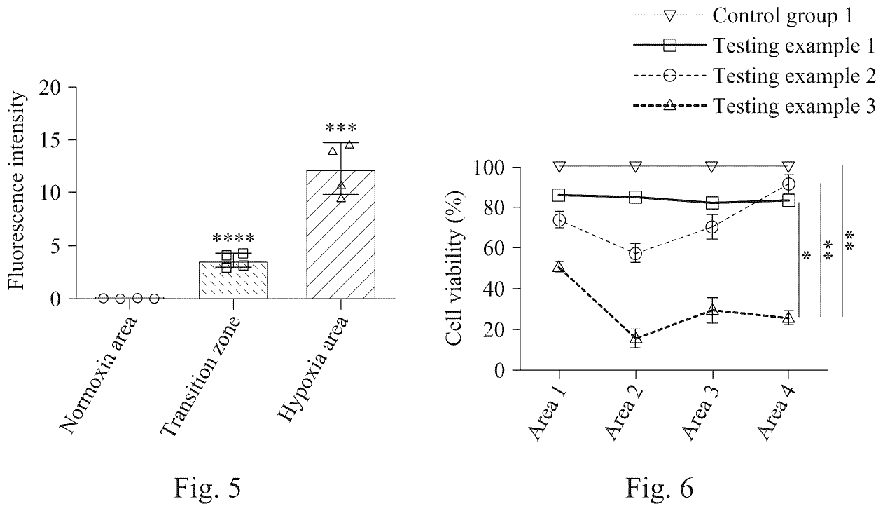

Reference is made to FIG. 4 and FIG. 5. FIG. 4 shows the results of Western blotting analysis of the co-cultivation of 4T1 cells and K-BALB cells for 24 hours by the microenvironment-simulated cell culture systems of Example 1 to Example 3 (hereafter referred to as “Example 1”, “Example 2” and “Example 3”). FIG. 5 shows the analysis results of hypoxia signal quantification in different areas of the cell culture chamber of the microenvironment simulated cell culture system of Example 2. As shown in FIG. 4, the expressions of HIF 1-α in the hypoxia areas of Example 1 and Example 2 increase, and the largest difference in expression is shown in Example 2. Further, in FIG. 5, the definition of the fluorescence intensity is the relative fluorescence intensity calculated based on the fluorescence intensity in the normoxia area as 1. As shown in FIG. 5, the hypoxia signals gradually increase along the direction from the normoxia area, the transition zone to the hypoxia area and have a significant increase in the hypoxia area.

As shown in the aforementioned results, the oxygen concentration gradient can be established in the cell culture chamber of the microenvironment-simulated cell culture system of the present disclosure, and it has the potential for use in relevant clinical trials.

III. Analysis of the Effects of the Gradient of Small Molecule Drugs on Cytotoxicity

In the present test, 4T1 cells and K-BALB cells are co-cultured in the microenvironment-simulated cell culture system of Example 2 so as to observe the effects of the small molecule drugs on 4T1 cells and K-BALB cells after being diffused from the cell culture medium to the cell culture chamber. In the present test, Control group 1 is performed with the cell culture medium without any drug, Testing example 1 is performed with the cell culture medium including Gemcitabine, Testing example 2 is performed with the cell culture medium including Galunisertib (an inhibitor of TGF-β1), and Testing example 3 is performed with the cell culture medium including Gemcitabine and Galunisertib. In detail, Gemcitabine is a synthetic cytosine derivative clinically used for the cancer chemotherapy, and it has the advantages of a strong radiosensitization effect and less toxic side effects. Further, Galunisertib, which is an inhibitor of the cytokine TGF-β1 closely related to the biochemical pathway of drug resistance, is further used in the present test, and Gemcitabine and Galunisertib are combined to use so as to observe the cells after being treated with the aforementioned combination.

In the present test, the concentration of Gemcitabine in the cell culture medium is 100 μM, and the concentration of Galunisertib in the cell culture medium is 100 μM. At the same time, the cell viabilities of 4T1 cells and K-BALB cells in different areas are further analyzed, and 4T1 cells and K-BALB cells are stained with the propidium iodide so as to observe the effects of different drug combinations on 4T1 cells and K-BALB cells cultured in the cell culture chamber of the cell culture chip of the microenvironment-simulated cell culture system of the present disclosure.

Reference is made to FIG. 6, which shows the analysis results of cell viability in different areas of the cell culture chamber after culturing for 24 hours with different drug combinations. As shown in FIG. 6, the cell viabilities of Area 1 to Area 4 of Testing example 1 are not significantly different there between, but the cell viabilities of Area 1 to Area 4 of Testing example 3 are significantly lower than that of other testing examples. Further, in Testing example 3, the cell viability of Area 4 is significantly lower than that of Area 1. Accordingly, the concentration gradient of drugs can be established in the cell culture chamber of the cell culture chip of the microenvironment-simulated cell culture system of the present disclosure, and can be used to simulate the growth state of tumor cells during drug administration.

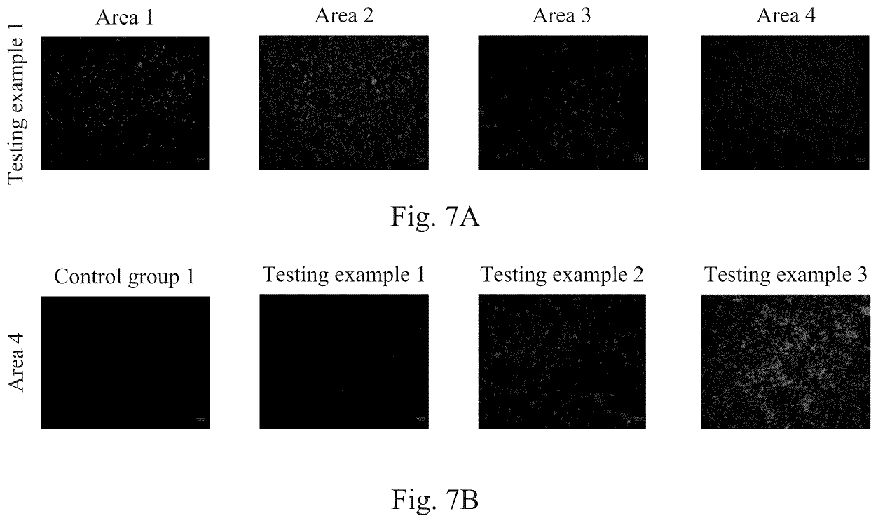

Reference is further made to FIG. 7A and FIG. 7B. FIG. 7A shows the staining results of the propidium iodide in different areas of the cell culture chamber of Testing example 1, and FIG. 7B shows the staining results of the propidium iodide in Area 4 of the cell culture chambers of Testing example 1 to Testing example 4. As shown in FIG. 7A, signals of the propidium iodide (red) in Area 1 and Area 2 of Testing example 1 are significantly higher than that of Area 3 and Area 4. Accordingly, it is shown that the concentrations of Gemcitabine in Area 1 and Area 2 are higher, so that the apoptosis of 4T1 cells and K-BALB cells can be induced, and the inhibition of apoptosis increase in the hypoxia area (that is, Area 3 and Area 4). Further, as shown in FIG. 7B, when lonely comparing the intensities of the propidium iodide signal in Areas 4, which is with the lowest oxygen concentration, of Testing example 1 to Testing example 3, the intensity of the propidium iodide signal of Testing example 3, which has a better drug treating performance in FIG. 6, is significantly higher than that of other examples. Accordingly, it is shown that different drug combinations can still have different effects on the cells in the areas with different oxygen concentrations in the cell culture chamber, and the results of the aforementioned tests are highly similar to the performance of the tumor microenvironment in clinical. Therefore, the microenvironment-simulated cell culture system of the present disclosure has an excellent ability to be used to study the interaction between drugs and oxygen gradients in the tumor microenvironment, and has excellent potential for clinical application.

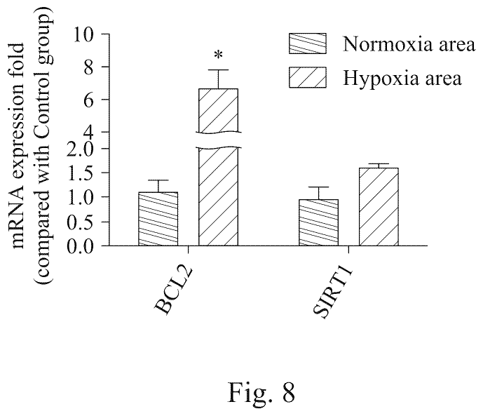

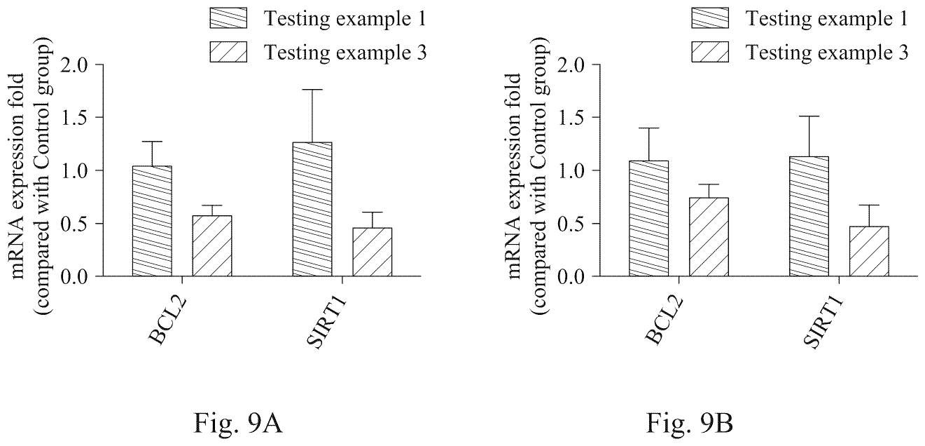

Reference is further made to FIG. 8, FIG. 9A and FIG. 9B simultaneously. FIG. 8 shows the quantification results of mRNA expression in the normoxia area and the hypoxia area of the cell culture chamber of Testing example 1, FIG. 9A shows the quantification results of mRNA expression in the normoxia areas of the cell culture chambers of Testing example 1 and Testing example 3, and FIG. 9B shows the quantification results of mRNA expression in the hypoxia areas of the cell culture chambers of Testing example 1 and Testing example 3. In detail, in the present test, qPCR is simultaneously used to measure the expression of mRNA of proteins related to apoptosis and drug resistance in the areas with different oxygen concentrations in the cell culture chamber of Testing example 1 and Testing example 3, wherein BCL2 is an apoptosis-regulating protein that can regulate cell death by inhibiting or inducing apoptosis, while SIRT1 can inactivate the drug by removing the acetyl group of the target protein.

As shown in FIG. 8, both of the mRNA expressions of BCL2 and SIRT1 in the hypoxia area of Testing example 1 are higher than that of the normoxia area. However, as shown in FIG. 9A and FIG. 9B, after treating with Galunisertib adjunctively, the mRNA expressions of BCL2 and SIRT1 significantly decrease in the normoxia area and the hypoxia area. Accordingly, it is shown that the microenvironment-simulated cell culture system of the present disclosure can indeed be used to simulate the effects of small molecule drugs on tumors, and it is consistent with the current clinical research results and has application potential in the relevant market.

IV. Analysis of the Expressions of Immune or Inflammatory Response-Related Proteins

In the present test, 4T1 cells and K-BALB cells are co-cultured in the microenvironment-simulated cell culture system of Example 2 so as to observe the mRNA expression of the proteins related to the immunity or inflammatory responses in different areas of the cell culture chamber. In the present test, Control group 2 is performed by culturing K-BALE cells using the cell culture medium without any drug; Testing example 4 is performed by co-culturing 4T1 cells and K-BALB cells using the cell culture medium without any drug, and then K-BALB cells are separated by magnetic beads for analysis; Testing example 5 is performed by co-culturing 4T1 cells and K-BALB cells using the cell culture medium including 50 μM Galunisertib, and then K-BALE cells are separated by magnetic beads for analysis; and Testing example 6 is performed by co-culturing 4T1 cells and K-BALB cells using the cell culture medium including 50 μM AZD-1480 (JAK1/2 inhibitor), and then K-BALB cells are separated by magnetic beads for analysis. Further, Control group 3 is performed by co-culturing 4T1 cells and K-BALB cells using the cell culture medium without any drug, and then 4T1 cells are separated by magnetic beads for analysis; Testing example 7 is performed by co-culturing 4T1 cells and K-BALB cells using the cell culture medium including 50 μM Galunisertib, and then 4T1 cells are separated by magnetic beads for analysis; and Testing example 8 is performed by co-culturing 4T1 cells and K-BALB cells using the cell culture medium including 50 μM AZD-1480, and then 4T1 cells are separated by magnetic beads for analysis. After culturing for 24 hours, the expressions of the immune or the inflammatory response-related proteins in the normoxia area and the hypoxia area of the cell culture chamber of each of Control group 2, Control group 3 and Testing example 4 to Testing example 8 are further measured.

In particular, the leukemia inhibitory factor (“LIF” hereafter) is a cytokine belonging to the interleukin 6 class cytokine and can affect the cell by inhibiting the differentiation thereof; the transforming growth factor-β1 (“TGF-β1” hereafter) can regulate the growth, the proliferation and the differentiation of the cell as well as regulate whether the apoptosis happens or not; the programmed cell death ligand 1 (“PD-L1” hereafter) is an important regulatory protein for the initiation of immune function in vivo; the collagen I (“col-I” hereafter) is associated with the differentiation of tumor or the inflammatory response; and the interleukin-1 (“IL-1” hereafter) plays an important role in controlling of immune and inflammatory responses.

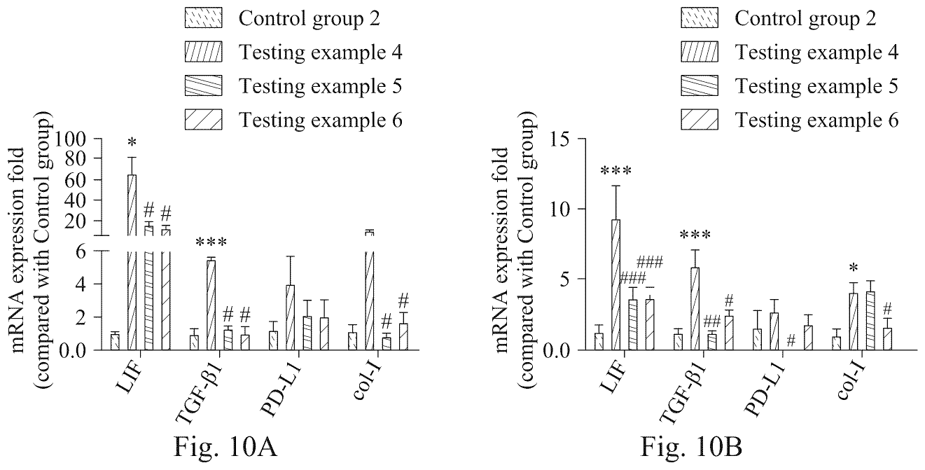

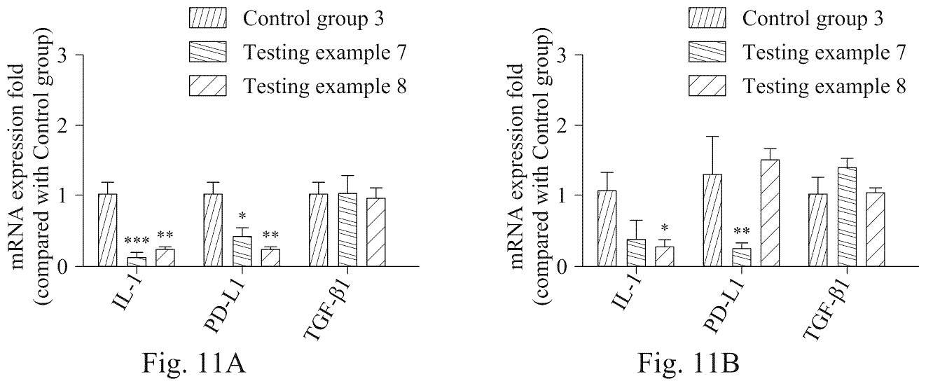

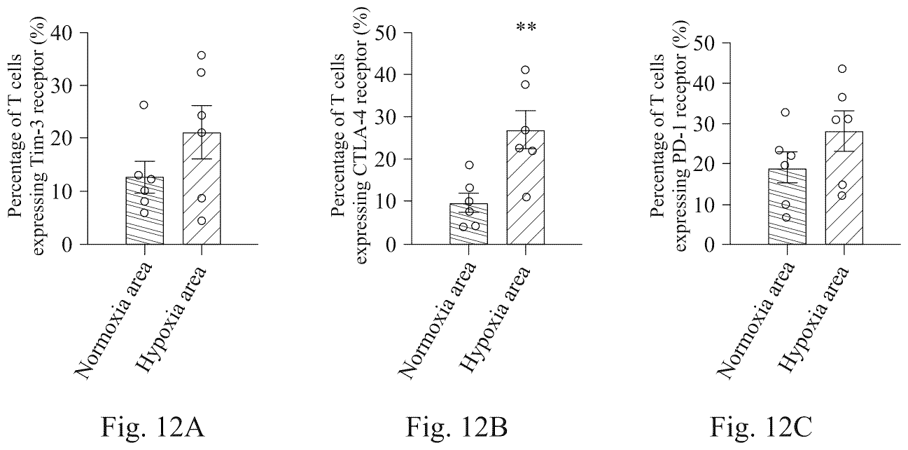

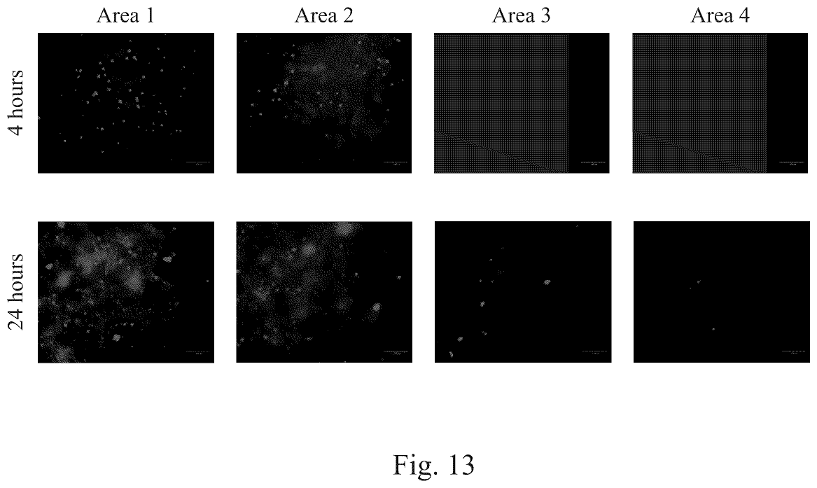

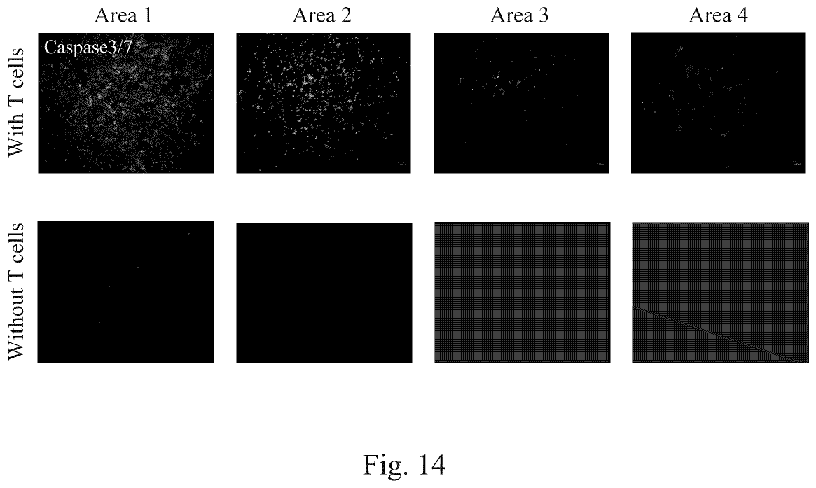

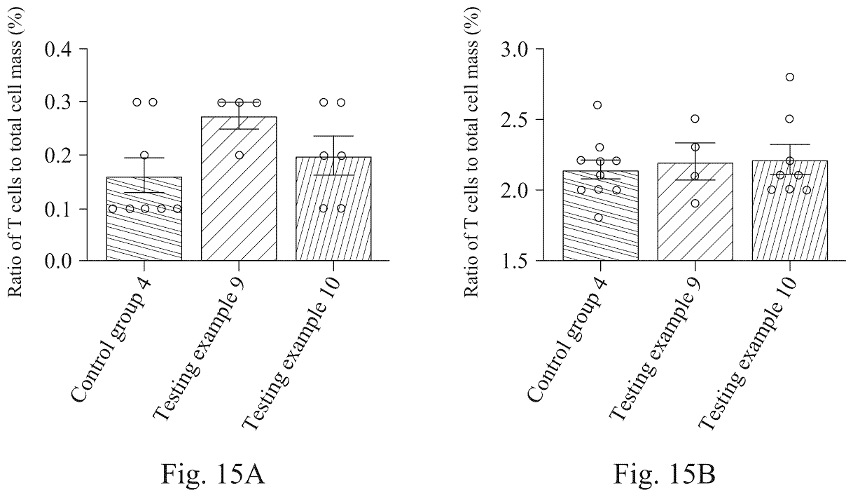

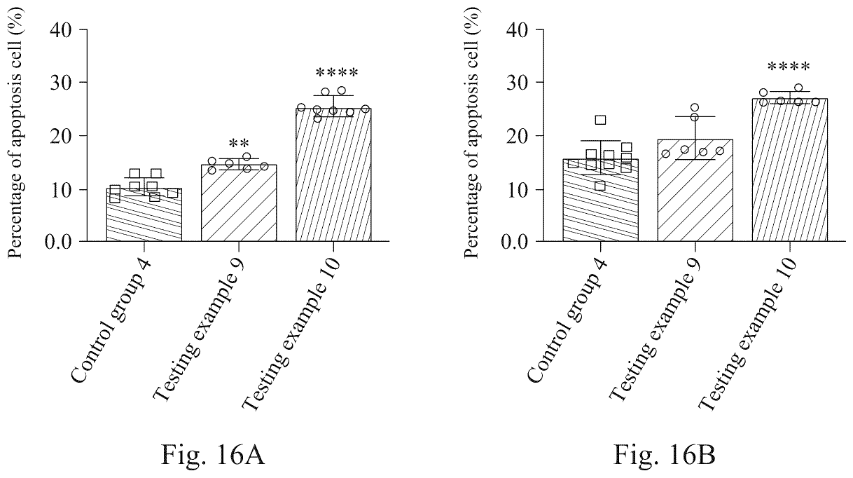

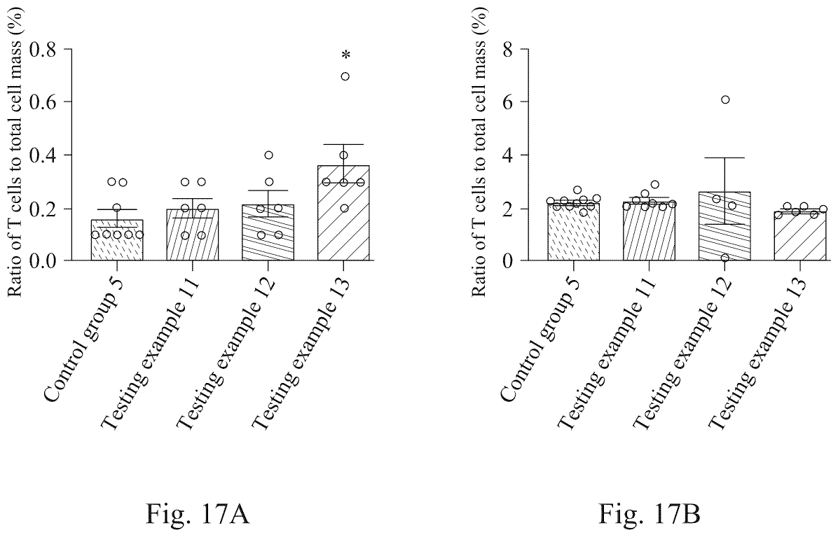

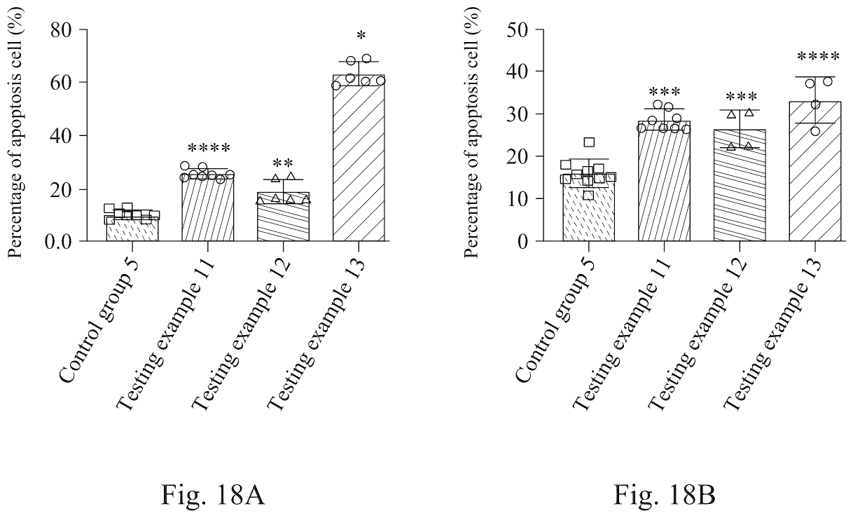

Reference is made to FIG. 10A, FIG. 10B, FIG. 11A and FIG. 11. FIG. 10A shows the quantification results of mRNA expression of the K-BALB cells in the normoxia areas of the cell culture chambers of Control group 2 and Testing example 4 to Testing example 6, FIG. 10B shows the quantification results of mRNA expression of the K-BALB cells in the hypoxia areas of the cell culture chambers of Control group 2 and Testing example 4 to Testing example 6, FIG. 11A shows the quantification results of mRNA expression of 4T1 cells in the normoxia areas of the cell culture chambers of Control group 3, Testing example 7 and Testing example 8, and FIG. 11B shows the quantification results of rnRNA expression of 4T1 cells in the hypoxia areas of the cell culture chambers of Control group 3, Testing example 7 and Testing example 3. In FIG. 10A, FIG. 10B, FIG. 11A and FIG. 11B, “*” represents that the statistical data is obtained by being compared with the data of Control group 2 or Control group 3, and “#” represents that the statistical data is obtained by being compared with the data of Testing example 4.

In detail, when K-BALB cells are cultured in the microenvironment-simulated cell culture system of Example 2 alone, some of the signals of fibrosis and inflammation will express in the hypoxia area of the cell culture chamber. Further, when 4T1 cells are cultured in the microenvironment-simulated cell culture system of Example 2 alone, the signals of inflammation will express in the hypoxia area of the cell culture chamber. However, as shown in FIG. 10A and FIG. 10B, after separating K-BALB cells of Testing example 4 to Testing example 6 by the magnetic beads and then analyzing by qPCR, all of the mRNA expressions of LIF, TGF-β1, PD-L1 and col-I of K-BALB cells in the normoxia area and the hypoxia area of the cell culture chamber of Testing example 4 significantly increase compared with that of Control group 2, and the mRNA expressions thereof decrease along with the application of Galunisertib or AZD-1480 and have different amounts correspondingly. Further, as shown in FIG. 11A and FIG. 11B, after separating 4T1 cells of Testing example 7 and Testing example 8 by the magnetic beads and then analyzing by qPCR, the mRNA expressions of IL-1 and PD-L1 in the normoxia area and the hypoxia area of the cell culture chamber of Testing example 7 and Testing example 8 significantly decrease compared with that of Control group 3, but both of the expressions of TGF-β1 of Testing example 7 and Testing example 8 are comparable to that of Control group 3. Accordingly, it is shown that the cells in the normoxia area and the hypoxia area of the cell culture chamber of the microenvironment-simulated cell culture system of the present disclosure can have different performances of the immune response or the inflammatory response, and the microenvironment-simulated cell culture system of the present disclosure can be used to simulate the effect of drugs on tumors and has application potential in the relevant market.

V. Analysis of the Content of T Cells in the Cell Culture Chip and the Expression Thereof

In the present test, 4T1 cells, K-BALB cells and T cells expressing CD3 are co-cultured in the microenvironment-simulated cell culture system of Example 2 so as to analyze the survival state of T cells in the microenvironment-simulated cell culture system of the present disclosure, and then the feasibility for use in immunotherapy research of the microenvironment-simulated cell culture system of the present disclosure is assessed. In the present test, T cells are transferred to the cell culture chamber along with the cell culture medium driven by the fluid driving member so as to simulate the state that T cells enter the tumor from the circulatory system during the growth of tumor in the real state. Then, after culturing for 24 hours, the expressions of the receptors associated with T-cell depletion, namely T-cell immunoglobulin domain and mucin domain-3 (“Tim-3” hereafter), the cytotoxic T lymphocyte associated antigen-4 (“CTLA-4” hereafter) and the programmed cell death protein-1 (“PD-1” hereafter), are analyzed, and the expressions of apoptosis-related protein, Caspase 3/7, in different areas of the cell culture chamber are analyzed, simultaneously.

Reference is made to FIG. 12A, FIG. 12B and FIG. 12C. FIG. 12A shows the analysis results of the percentage of T cells expressing Tim-3 receptor in the normoxia area and the hypoxia area of the cell culture chamber, FIG. 12B shows the analysis results of the percentage of T cells expressing CTLA-4 receptor in the normoxia area and the hypoxia area of the cell culture chamber, and FIG. 12C shows the analysis results of the percentage of T cells expressing PD-1 receptor in the normoxia area and the hypoxia area of the cell culture chamber. In particular, T cells are transferred to the cell culture chamber along with the cell culture medium driven by the fluid driving member, and as shown in the results of the pre-analysis of the infiltration of T cells, the amount of T cells in the normoxia area is significantly higher than that in the hypoxia area. Further, as shown in FIG. 12A to FIG. 12C, the proportions of T cells expressing the receptors of Tim-3, CTLA-4 and PD-1 to total cells in the hypoxia area are significantly increased. Accordingly, it is shown that the immunotherapy resistance in the hypoxia area can be observed in the microenvironment-simulated cell culture system of the present disclosure.

Reference is further made to FIG. 13 and FIG. 14. FIG. 13 shows the staining results of T cells in different areas of the cell culture chamber after being cultured for 4 hours and 24 hours, and FIG. 14 shows the staining results of apoptosis signaling-related proteins in different areas of the cell culture chamber. As shown in FIG. 13, after culturing for 4 hours and 24 hours, the signals of T cells (red) gradually decrease from Area 1 to Area 4 of the cell culture chamber, and thus it is shown that the gradient of the immune cells in the actual environment can be effectively simulated by the cell culture chip of the microenvironment-simulated cell culture system of the present disclosure at different times. Further, as shown in FIG. 14, in the presence of T cells, the expressions of Caspase 3/7 (green) also gradually decrease from Area 1 to Area 4 of the cell culture chamber, and thus it is shown that the apoptosis of cells is suppressed in the hypoxia area. Furthermore, under the condition without T cells, the expressions of Caspase 3/7 are approximately the same in Area 1 to Area 4 of the cell culture chamber. Accordingly, it is shown that the microenvironment-simulated cell culture system of the present disclosure has the potential to apply to the immunotherapy research of cancer and has excellent clinical application potential.

VI. Analysis of the Effects of the Immune Checkpoint Inhibitor and the Anticancer Drug Therapy

In the present test, 4T1 cells, K-BALB cells and T cells expressing CD3 are co-cultured in the microenvironment-simulated cell culture system of Example 2 so as to analyze the survival state of 4T1 cells, K-BALB cells and T cells after being treated with the immune checkpoints inhibitor and anticancer drugs, and then the feasibility for use in immunotherapy research of the microenvironment-simulated cell culture system of the present disclosure is assessed.

In the present test, Control group 4 is performed with the cell culture medium without any drug, Testing example 9 is performed by treating the cells in the cell culture chamber with low dose anti-PD-1 antibody at 100 ng/mL, and Testing example 10 is performed by treating the cells in the cell culture chamber with high dose anti-PD-1 antibody at 1000 ng/mL. Further, Control group 5 is performed with the cell culture medium without any drug, Testing example 11 is performed by treating the cells in the cell culture chamber with 1000 ng/mL of anti-PD-1 antibody, Testing example 12 is performed by treating the cells in the cell culture chamber with 50 μM of Galunisertib, and Testing example 13 is performed by treating the cells in the cell culture chamber with 1000 ng/mL of anti-PD-1 antibody as well as 50 μM of Galunisertib.

Reference is made to FIG. 15A, FIG. 15B, FIG. 16A and FIG. 16B. FIG. 15A shows the analysis results of the ratio of T cells to the total cell mass in the hypoxia area of the cell culture chamber after being treated with different doses of anti-PD-1 antibody, FIG. 15B shows the analysis results of the ratio of T cells to the total cell mass in the normoxia area of the cell culture chamber after being treated with different doses of anti-PD-1 antibody, FIG. 16A shows the analysis results of the percentage of apoptosis in the hypoxia area of the cell culture chamber after being treated with different doses of anti-PD-1 antibody, and FIG. 16B shows the analysis results of the percentage of apoptosis in the normoxia area of the cell culture chamber after being treated with different doses of anti-PD-1 antibody.

As shown in FIG. 15A and FIG. 15B, after treating by high dose of anti-PD-1 antibody, the infiltration amount of T cells in the hypoxia area of the cell culture chamber of Testing example 9 is higher than that of Testing example 10. Further, in the normoxia area, there is no significant difference between the infiltration amount of T cells in the cell culture chamber of Testing example 9 and that of Control group 4 regardless of whether the low dose of anti-PD-1 antibody or the high dose of anti-PD-1 antibody is administered. However, as shown in FIG. 16A and FIG. 16B, the proportions of apoptosis cells in both of the hypoxia area and the normoxia area are larger than that of Testing example 9 and Testing example 10, wherein the proportions of apoptosis cells in both of the hypoxia area and the normoxia area of Testing example 10 are largest. Accordingly, it is shown that the microenvironment-simulated cell culture system of the present disclosure has the potential to apply to the immunotherapy research of cancer.

Reference is further made to FIG. 17A, FIG. 17B, FIG. 18A and FIG. 18B. FIG. 17A shows the analysis results of the ratio of T cells to the total cell mass in the hypoxia area of the cell culture chamber after being treated with different drugs, FIG. 17B shows the analysis results of the ratio of T cells to the total cell mass in the normoxia area of the cell culture chamber after being treated with different drugs, FIG. 18A shows the analysis results of the percentage of apoptosis in the hypoxia area of the cell culture chamber after being treated with different drugs, and FIG. 18B shows the analysis results of the percentage of apoptosis in the normoxia area of the cell culture chamber after being treated with different drugs.

As shown in FIG. 17A and FIG. 17B, the infiltration amount of T cells in the hypoxia area of Testing example 13 is the highest, but in the normoxia area, the infiltration amounts of T cells of Testing example 11 to Testing example 13 have no difference compared with that of Control group 5. However, as shown in FIG. 18A and FIG. 18B, all of the proportions of apoptosis 4T1 cells in the hypoxia area and the normoxia area of the cell culture chamber of Testing example 11 to Testing example 13 are larger than that of Control group 5, wherein the proportion of apoptosis cells of Testing example 13 is the highest. Accordingly, it is shown that anti-PD-1 antibody and the Galunisertib have an excellent synergistic inhibitory effect to 4T1 cells.

To sum up, in the microenvironment-simulated cell culture system of the present disclosure, not only the cells can be cultured in the three-dimensional space of the cell culture chamber so as to simulate the hypoxia condition of the area far away from the circulatory system in the tumor for drug screening or immunoassay testing, but also the states that the tumor is rich in the extracellular matrix and the hyperplasia of the connective tissues can be simulated by supplying the extracellular matrix fluids such as collagen. Therefore, the microenvironment-simulated cell culture system of the present disclosure can be used for anticancer drug screening and related treatment tests for different types of cancer, and has clinical application potential.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. A microenvironment-simulated cell culture system, comprising:
a cell culture chip, comprising:
a mainbody;
a cell culture chamber disposed in the mainbody and comprising a first side portion and a second side portion, wherein the first side portion and the second side portion are respectively disposed on two ends of the cell culture chamber along a long axis of the mainbody;
two fluid delivery ports separately disposed on the mainbody and respectively connected to the cell culture chamber; and
a sample loading well disposed on the mainbody and connected to the cell culture chamber;
a fluid storage device pipe-connected to the cell culture chip, wherein the fluid storage device is connected to the cell culture chamber by one of the fluid delivery ports; and
a fluid driving member pipe-connected to the fluid storage device and the cell culture chip, wherein the fluid driving member is connected to the cell culture chamber by the other one of the fluid delivery ports;
wherein the cell culture chamber is substantially a long-stripped slot, two long sides of the cell culture chamber are parallel to the long axis of the mainbody, and a length ratio of a short side of the cell culture chamber to one of the long sides of the cell culture chamber is 1:1 to 1:4.

2. The microenvironment-simulated cell culture system of claim 1, wherein:
the cell culture chip is a multi-layers structure and comprises a first base plate, a second base plate, a third base plate, a fourth base plate, a fifth base plate, a sixth base plate and a seventh base plate;
the first base plate has a first surface, and the two fluid delivery ports are separately opened on the first surface, wherein the first base plate, the second base plate, the third base plate and the fifth base plate are stacked in sequence to form a fluid channel, and the fluid channel is connected to the two fluid delivery ports and the cell culture chamber;
the fourth base plate has a second surface, and the sample loading well is opened on the second surface, wherein the fourth base plate and the fifth base plate are stacked in sequence to form a loading channel, and the loading channel is connected to the sample loading well and the cell culture chamber; and
the fifth base plate, the sixth base plate and the seventh base plate are stacked in sequence to form the cell culture chamber.

3. The microenvironment-simulated cell culture system of claim 2, wherein:
the first base plate, the second base plate and the third base plate are stacked in sequence to form a first covering unit, and the first covering unit covers the first side portion; and
the fourth base plate covers the second side portion.

4. The microenvironment-simulated cell culture system of claim 2, wherein the first base plate, the second base plate, the third base plate, the fourth base plate, the fifth base plate, the sixth base plate and the seventh base plate are made of an impermeable material.

5. The microenvironment-simulated cell culture system of claim 4, wherein the impermeable material is polyethylene terephthalate, acrylic, polycarbonate, polystyrene or glass.

6. The microenvironment-simulated cell culture system of claim 4, wherein the impermeable material is a transparent material.

7. The microenvironment-simulated cell culture system of claim 1, wherein the two fluid delivery ports are disposed along a direction parallel to the short side of the cell culture chamber.

8. The microenvironment-simulated cell culture system of claim 1, wherein the fluid storage device is for storing a cell culture medium, and the fluid driving member is for continuously driving the cell culture medium to be transferred from the fluid storage device to the cell culture chamber through the one of the fluid delivery ports and then to be removed from the cell culture chamber through the other one of the fluid delivery ports.

9. The microenvironment-simulated cell culture system of claim 8, wherein the fluid driving member is a peristaltic pump.

10. The microenvironment-simulated cell culture system of claim 1, wherein the length ratio of the short side of the cell culture chamber to the one of the long sides of the cell culture chamber is 1:2.
